(19) United States
(12) Patent Application Publication
(10) Pub. No.: US 2005/0187528 A1
(43) Pub. Date: Aug. 25, 2005

(54) Self-cleaning apparatus for material transfer, collection and disposal

(75) Inventor: Nicholas J. Berg, Milford, DE (US)

Correspondence Address: STERNE, KESSLER, GOLDSTEIN & FOX PLLC, 1100 NEW YORK AVENUE, N.W., WASHINGTON, DC, 20005, US

(73) Assignee: ENT By The Sea, LLC, Milford, DE (US)

(21) Appl. No.: 10/785,065

(22) Filed: Feb. 25, 2004

(51) Int. Cl. A61M 1/00

(52) U.S. Cl. 604/317

(57) ABSTRACT

The present invention is a self-cleaning apparatus for transferring, collecting and disposing of a material. While the apparatus has specific utility in the medical and surgical environment for disposing of bodily wastes such as blood, mucous, wax, cauterized tissue and the like, the apparatus can also be employed to collect and transfer or dispose of any material, constituent or product. The apparatus includes a material transfer hose, a material collection chamber, a vacuum source for drawing material into the material collection chamber through the material transfer hose, an optional pump for emptying the material collection chamber, and a cleaning fluid chamber which receives the material transfer hose to permit a cleaning fluid to be drawn into the material transfer hose to clean the hose, as well as the remaining components of the apparatus. The optional disposal pump can be activated automatically to permit non-interrupted transfer, collection and disposal of a material to a drain.

### CROSS-REFERENCE TO RELATED APPLICATIONS

Not applicable.

### STATEMENT REGARDING FEDERALLY-SPONSORED RESEARCH AND DEVELOPMENT

Not applicable.

### REFERENCE TO MICROFICHE APPENDIX/SEQUENCE LISTING/TABLE/COMPUTER PROGRAM LISTING APPENDIX (Submitted on a Compact Disc and an Incorporation-By-Reference of the Material on the Compact Disc)

Not applicable.

### BACKGROUND OF THE INVENTION

1. Field of the Invention

The present invention relates to a self-cleaning apparatus for the collection, transfer and disposal of material. The apparatus has particular utility in collecting, transferring and disposing of medical or surgical waste, but also finds application in any field involving the collection, transfer and disposal of any waste material, constituent or product.

2. Background Art

During surgical and other medical procedures, blood, mucous, wax and other potentially infectious fluids and materials are collected and removed from the surgical site or procedure area. Such fluids and materials must be eventually disposed of without undue risk of infection to health-care workers by pathogens present therein, or contamination of previously sterile surfaces and instruments.

Various techniques are known in the art for collecting bodily fluids and materials in conjunction with surgical and medical procedures. The collection vessels vary from simple plastic bags to elaborate, electronically controlled processing equipment (e.g., the Neptune Waste Management System by Stryker Instruments).

In conventional fluid waste collection and disposal systems, blood and other fluids or materials are suctioned from a surgical or therapeutic site on a patient and are collected in one or more plastic canisters. The canisters are somewhat small and are typically included as part of a larger free-standing portable unit located within an operating room or doctor's office. Thus, the portable unit is space-consuming and must be maneuvered from one area to another for use and to dispose of the fluid waste.

When a fluid waste collection canister is filled during the course of a surgical or medical procedure, the canister is removed from the portable unit and emptied (if the canister is re-usable) or the canister is replaced with a new one (if the canister is for disposable use). Fluid waste from the canister is either emptied directly into a sink and/or drain connected to a sewer line, emptied into a larger container and transported off-site, or emptied into a larger container and transported through the facility to a central collection location, where the fluid waste is either chemically treated and disposed of in the sewer or incinerated on-site. Obviously, this handling of fluid waste by a health-care worker while transporting canisters to and from the portable unit to the disposal area, increases the risk of exposure to pathogens within the fluid waste and possible contamination of the type mentioned above. Cleaning of the fluid waste collection canisters, in the event that they are re-usable, only further increases the risk of infection and contamination.

In addition to the preceding drawback, this technique requires that the surgical or medical procedure be interrupted to empty the canister when it has been filled, even if the procedure is not complete. Furthermore, the use of disposable canisters can be costly and requires that an inventory of canisters be kept on hand and available at all times.

U.S. Pat. No. 4,957,491 to Parker discloses a combination fluid collection and disposal apparatus for collecting and disposing fluid aspirated from a patient during a surgical procedure. The apparatus comprises a collection unit for collecting fluid and a separate treatment unit coupled to the collection unit to remove and dispose of fluid from the collection unit. The collection unit comprises a reservoir for the temporary storage of fluids aspirated from the patient, a vacuum port for connecting the collection unit to a vacuum source, and a vacuum line connecting the vacuum port to the reservoir. The collection unit also comprises at least one suction port adapted for connection to a suction tube for aspirating fluids from the patient, a suction line connecting the suction port to the reservoir, and a drain generally adjacent the bottom of the reservoir. The treatment unit comprises a washing fluid port adapted for connection to a source of washing fluid and means for coupling the treatment unit to the suction port for providing washing fluid through the suction port, suction line and reservoir for cleaning all of the same. The treatment unit also comprises means for coupling the treatment unit to the drain for removing the accumulated aspirated fluid and the washing fluid from the reservoir. While the device of the Parker patent can be used to aspirate and collect fluid waste from a surgical or therapeutic site, it requires that aspiration be interrupted when the reservoir of the collection unit has been filled to capacity. In addition, the collection unit must be wheeled to the location of the treatment unit so that fluid waste within the reservoir can be emptied and the suction tube, suction line, and reservoir cleaned by connecting it to the washing fluid port of the collection unit. Upon emptying (and optionally cleaning) the collection unit, the collection unit must be wheeled back to the surgical or therapeutic site to continue with the aspiration required by the procedure. Obviously, this interruption in the procedure is undesirable.

Attempts to avoid transporting canisters containing fluid waste from one location to another have been made. U.S. Patent Application Publication No. U.S. 2002/0082569 A1 to Wildman discloses a low exposure waste disposal suction system for collecting and disposing of fluid waste from a patient undergoing a medical procedure in an operating room comprising a tank and a conduit. The tank is positioned outside of the operating room. The conduit extends from the tank and into the operating room. Fluid waste from the patient is suctioned through the conduit and collected in the tank. When the tank is filled, a valve is activated and the fluid waste collected in the tank is emptied into a drainage system. The system further comprises a cleaning unit in fluid communication with the tank for cleaning the tank. While the Wildman publication requires no canisters, the tank of the device must still be emptied when it reaches capacity, which could conceivably result in an interruption of the procedure. In addition, it is not possible to clean all components of the system, particularly the conduit through which waste is suctioned, without removing and re-attaching the conduit to a different component of the system. This extra handling step subjects the health care worker to possible exposure to pathogens possibly contained within the fluid waste and could also result in contamination of an otherwise sterile environment.

Still another system for disposing of both waste liquid and smoke, typically in, but not limited, to a surgical environment, is disclosed in U.S. Pat. No. 6,180,000 B1 to Wilbur et al. The smoke extraction system and liquid waste collection system of the patent are connected to supply the materials collected thereby to a treatment and disposal system. The treatment system includes a plurality of heat exchangers for controlling the temperature of the waste material during the treatment process. These three systems are combined into an integrated system, wherein the treated waste can be safely collected and disposed to a sewer by a pump within the system connected to a drain. While the integrated system of the patent provides for decontamination and sterilization of liquid waste and smoke, it does not provide for cleaning of the components of the system to prevent contamination between surgical procedures.

Thus, there is a need for a material collection and disposal apparatus which reduces the risk of exposure of a health care worker to harmful agents or pathogens, by dispensing of reusable or disposable canisters.

There is another need for a material collection and disposal apparatus capable of continuously collecting and disposing of material without interrupting the surgical or medical procedure to empty or drain a collection unit.

There is still another need for a material collection and disposal apparatus that allows for cleaning of all parts of the apparatus, including the material interface, to prevent infection or contamination between uses.

There is yet another need for a material collection and disposal apparatus that is easily maintained and has few components.

The need also exists for a material collection and disposal system that can be easily connected to a drain or sewer line to permit disposal of the material, without risk to the health-care worker or sterility of the surgical environment.

There is yet another need for a material collection and disposal apparatus which permits collection and disposal of the material, as well as cleaning of the components of the apparatus, in an integrated system.

### BRIEF SUMMARY OF THE INVENTION

With the above needs in mind, the present invention is a self-cleaning apparatus for transferring, collecting and disposing of a material. While the apparatus has specific utility in the medical and surgical environment for disposing of bodily wastes such as blood, mucous, wax, cauterized tissue and the like, the apparatus can be employed to collect and transfer or dispose of any material, constituent or product. In one embodiment, the apparatus includes a material transfer hose, a material collection chamber, a vacuum source for drawing material into the material collection chamber through the material transfer hose, an optional pump for emptying the material collection chamber, and a cleaning fluid chamber which receives the material transfer hose to permit a cleaning fluid to be drawn into the material transfer hose by the vacuum source to clean the hose, as well as the material collection chamber and optional disposal pump. The optional disposal pump is activated to permit non-interrupted transfer, collection and disposal of material to a drain.

In another aspect, the invention is a self-cleaning apparatus for transferring, collecting and disposing of waste material from a patient. The apparatus comprises a waste material transfer hose, a waste material collection chamber, a vacuum source and a cleaning fluid chamber. The waste material transfer hose has an inlet and an outlet for transferring waste material from the patient to the apparatus. The waste material collection chamber has a collection chamber inlet in communication with the outlet of the waste material transfer hose and a collection chamber outlet. The vacuum source is connected to the waste material collection chamber by a vacuum line. The cleaning fluid chamber receives a cleaning fluid and is arranged to communicate with and receive the inlet of the waste material transfer hose. The cleaning fluid chamber is disposed upstream of the inlet of the waste material transfer hose to permit the cleaning fluid to be drawn into the waste material transfer hose and waste material collection chamber by the vacuum source, to clean the apparatus. The apparatus may comprise a float disposed within the vacuum line to prevent waste material within the waste material collection chamber from being drawn into the vacuum line by the vacuum source, when the waste material collection chamber is filled to capacity. The apparatus may also comprise a disposal pump in communication with the outlet of the waste material collection chamber to pump waste material out of the waste material collection chamber. The apparatus may further comprise a switch in electrical contact with the float and the disposal pump, such that when the float closes the vacuum line, the disposal pump is automatically activated to dispose of waste material in the waste material collection chamber through the collection chamber outlet. The disposal pump may be connected to a sewer drain.

In yet another aspect, the invention is an apparatus for transferring, collecting and disposing of a material comprising a material transfer hose, a material collection chamber, a vacuum source, a disposal pump, and a cleaning fluid chamber. The material transfer hose has an inlet and an outlet for transferring material to the apparatus. The material collection chamber has a collection chamber inlet in communication with the outlet of the material transfer hose and a collection chamber outlet. The vacuum source is connected to the material collection chamber by a vacuum line to draw material into the material collection chamber through the material transfer hose. The disposal pump is in communication with the collection chamber outlet to pump material out of the material collection chamber to dispose of material. The cleaning fluid chamber receives a fluid and is arranged to communicate with the inlet of the material transfer hose to permit cleaning fluid to be drawn into the inlet of the material transfer hose to clean the material transfer hose, material collection chamber and disposal pump.

In still another aspect, the invention is a method of cleaning a material transfer, collecting and disposal apparatus having at least a cleaning fluid chamber, a material transfer hose, a material collection chamber having an inlet in fluid communication with the material transfer hose and an outlet, and a vacuum source connected to the material collection chamber. The method comprises the steps of providing a cleaning fluid chamber with a cleaning fluid, inserting the material transfer hose in the cleaning fluid chamber, activating the vacuum source so that the cleaning fluid is drawn into the material transfer hose and the material collection chamber through the inlet of the material transfer hose to clean the material transfer hose and the material collection chamber. The method may also comprise the steps of de-activating the vacuum source and disposing of the cleaning fluid from the outlet of the material collection chamber after the material transfer hose and material collection chamber have been cleaned. The cleaning fluid also cleans the disposal pump as the cleaning fluid is pumped therethrough. The disposal step may be performed by a disposal pump in fluid communication with the outlet of the material collection chamber. The de-activating step may be performed by a switch activated when the material collection chamber is filled to capacity with the cleaning fluid.

### BRIEF DESCRIPTION OF THE DRAWINGS/FIGURES

The foregoing and other features and advantages of the present invention will be made apparent from the following detailed description of preferred embodiments of the invention, and the accompanying drawings in which:

FIG. 1 is a schematic view of the apparatus of the present invention;

FIG. 2 is a schematic view of the apparatus shown in FIG. 1 configured for self-cleaning;

FIG. 3 is a schematic view of an alternative embodiment of the present invention; and

FIG. 4 is schematic view of another embodiment of the invention.

### DETAILED DESCRIPTION OF THE INVENTION

A description of the preferred embodiment of the present invention appears below with reference to the above-described figures, where like reference numerals refer to identical or functionally similar structures or components. Also, in the figures, the left most digit of each reference number corresponds to the figure in which the reference number is first used. While specific configurations are discussed below, it should be understood by those skilled in the art that the description represents an illustration of the preferred embodiment, and that other configurations may be employed, either in whole or in part, without departing from the spirit and scope of the invention.

As noted above, the present invention is a self-cleaning apparatus for transferring, collecting and disposing of a material. While the apparatus has specific utility in the medical, dental and surgical environment for disposing of bodily wastes such as blood, mucous, wax, cauterized tissue and the like, the apparatus can also be employed to collect and transfer or dispose of any material, constituent or product. For example, it is envisioned that the apparatus of the present invention may be employed in the medical, surgical, dental and veterinary fields, as well as in the fields of agriculture, food processing, product manufacturing or any other area where the transfer, collection and disposal (or simply the collection and transfer) of a material from one location to another is desired.

Furthermore, while the description may refer to “waste” material, the material need not be waste or refuse, but may be any material, constituent or product that is capable of being transferred, collected and/or disposed of by the present invention. Also, with respect to the description of the apparatus as it relates to the transfer, collection and disposal of bodily waste, such waste may be liquid or solid, and may include but is not limited to blood, mucous, saliva, wax or cauterized tissue. In addition, the waste may also include non-bodily waste, including saline or other fluids introduced to the patient site during a procedure.

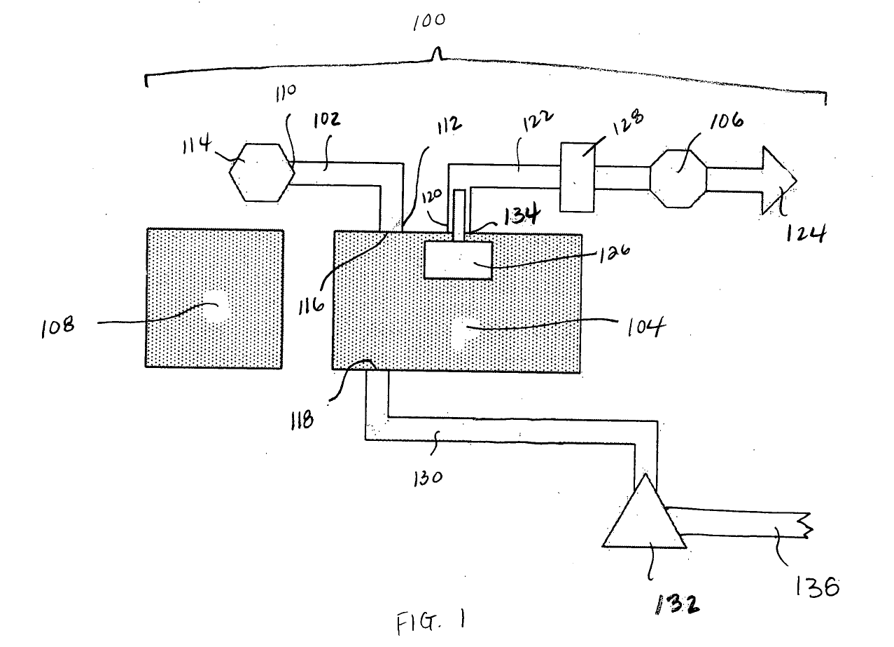

Referring now to FIG. 1, a self-cleaning apparatus for transferring, collecting and disposing of a waste or other material is shown at 100. Apparatus 100 comprises a material transfer hose 102, a material collection chamber 104, a vacuum source 106 and a cleaning fluid chamber 108. Material transfer hose 102 has a length extending between an inlet 110 and an outlet 112. Material transfer hose 102 may be constructed from rubber, plastic or other materials known in the art and capable of transferring material to the remaining components of the apparatus. The hose construction must be flexible to permit ease of use and its interior surface must be able to withstand the detergents and other agents of the cleaning fluid used to clean the material transfer hose and other components of the apparatus.

At its inlet 110, material transfer hose 102 is connected to an material interface 114 which receives the material from a material source (e.g., a surgical site on a patient). In the medical, surgical or dental fields, material interface may take the form of a nozzle, a suction instrument, a cauterizing probe with a suction port, or the like. Outlet 112 of material transfer hose 102 is disposed in fluid communication with material collection chamber 104 to deliver material transferred from the material source through the material interface and material transfer hose to the material collection chamber.

Material collection chamber 104 comprises a collection chamber inlet 116 which mates with outlet 112 of material transfer hose 102 and a collection chamber outlet 118. Material collection chamber 104 also has a vacuum opening 120 which is connected to vacuum source 106 to be discussed in more detail below. Material collection chamber 104 is essentially a tank which may be constructed to hold any volume and is intended to collect the material transferred into the apparatus by material transfer hose 102. Like material transfer hose 102, material collection chamber 104 should be constructed of a material, for example poly vinyl chloride (PVC), which is resistant to corrosion or erosion by the material contained therein, or the detergents of the cleaning fluid used to clean the components of the apparatus.

Vacuum source 106 is connected to material collection chamber 104 by a vacuum line 122 which engages with vacuum opening 120 of the material collection chamber. When activated, vacuum source 106 creates a negative pressure within material collection chamber 104 to draw material into the material collection chamber through material interface 114 and material transfer hose 102. Vacuum source 106 is activated by a switch (not shown) in electrical connection with a power source (not shown). A vacuum exhaust is provided at 124.

A float 126 is positioned within vacuum opening 120 to movably float therein. Normally, the float is positioned within, but does not obstruct, vacuum opening 120. However, when material collection chamber 104 is filled to capacity, the material within the chamber pushes float 126 into contact with a seat 134 to close vacuum line 122 to vacuum source 106, to prevent material from being drawn into the vacuum source, which could interfere with operation of the apparatus. A filter or fluid barrier 128 is positioned within vacuum line 122 between vacuum opening 120 and vacuum source 106 to prevent stray fluid or solid material from being drawn into the vacuum source when float 126 is not engaged with seat 134 and vacuum line 122 is open. Filter or fluid barrier 128 may be of the type sold by Millipore, model no. Millex FA 50, or any other structure capable of fulfilling the required function and known to those skilled in the art.

At outlet 118, material collection chamber 104 is attached to a disposal line 130. Disposal line 130 is located downstream of material collection chamber 104 and disposes of material contained therein. Disposal line 130 is connected to a drain 136 which may lead to a sewer or a larger material collection tank located distant to the apparatus. More particularly, disposal line 130 could be connected to the drain of a sink, dishwasher, shower, bathtub or any other piece of plumbing which leads away from the apparatus to a disposal area, such as a sewer or sump.

A valve 132 is positioned between material collection chamber 104 and drain 136, and within disposal line 130. Valve 132 is closed when vacuum source 106 is activated to prevent material within drain 136 or disposal line 130 from being drawn back into material collection chamber 104. Valve 132 is opened (manually or otherwise) to empty material collection chamber 104 when it is filled to capacity or at any other desirable time.

As previously discussed, apparatus 100 further includes a cleaning fluid chamber 108 positioned proximate to material transfer hose 102 of the apparatus. Cleaning fluid chamber 108 may be of any size, but should have a volume large enough to provide an adequate amount of cleaning fluid to the apparatus, to clean the components of the same. Like the other components of the invention, cleaning fluid chamber 108 should be constructed of a material capable of withstanding the detergents and other cleaning agents of the cleaning fluid received therein.

To use apparatus 100 of the present invention, a material interface 114 is attached to inlet 110 of material transfer hose 102. Material interface is brought into contact with the material to be transferred, collected and disposed of, and the apparatus is powered by activating power source 124. Upon activating the power source, vacuum source 106 generates a negative pressure within material collection chamber 104 via vacuum line 122. Material is drawn into material interface 114 by the vacuum and flows into material transfer hose 102 through inlet 110. From outlet 112 of material transfer hose 102, material is drawn into inlet 116 and collected within material collection chamber 104 of the apparatus. While the negative pressure within material collection chamber 104 is great enough to draw material into the chamber it is not great enough to prevent the material from flowing out of material collection chamber 104 through outlet 118. As the collected material flows out of outlet 118 (when valve 132 is open), disposal line 130 carries the material to drain 136 for ultimate disposal in a sewer or other disposal or treatment area. Thus, the apparatus of the present invention continuously transfers, collects and disposes of a material, without risk of contamination or interruption of the material transfer procedure. It does so using few parts and without the re-usable or disposable containers previously known in the art.

In the event that material within material collection chamber 104 is not emptied as quickly as it is drawn into the apparatus, and the material collection chamber fills to capacity, float 126 will engage seat 134 to close vacuum line 122 and discontinue transfer of material from the material source through the material interface and material transfer hose (as normally accomplished by vacuum source 106). Material will continue to flow out of material collection chamber 104 through disposal line 130 and into drain 136 when valve 132 is open, and as the level of material within material collection chamber 104 drops, float 126 will disengage with seat 134 to re-open the vacuum line and permit material to once again be drawn into the material interface and material transfer hose by the suction provided by vacuum source 106.

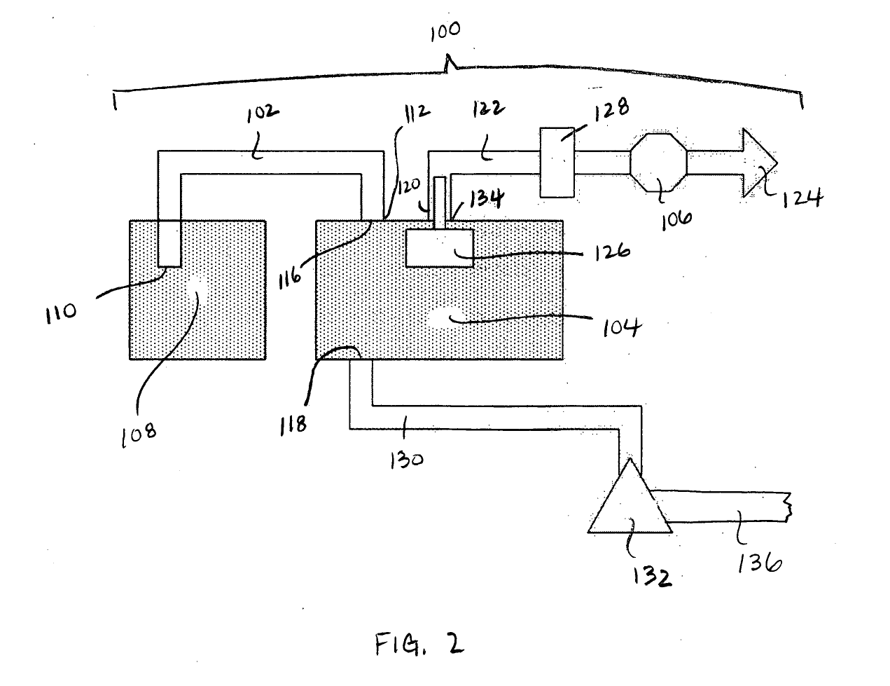

Upon transferring, collecting and disposing of a material, all components of apparatus 100 can be cleaned by utilizing integral cleaning fluid chamber 108. With reference now to FIG. 2, material interface 114 is removed from inlet 110 and material transfer hose 102 is inserted into cleaning fluid chamber 108. A suitable cleaning fluid is then poured into cleaning fluid chamber 108 and the power source activated. The suction generated by vacuum source 106 draws the cleaning fluid through material transfer hose 102 and into material collection chamber 104 to clean the same. After opening valve 132, and as the cleaning fluid flows out of material collection chamber 104 through outlet 118 to drain 136, disposal line 130 is also cleaned as the cleaning fluid passes therethrough. When the supply of cleaning fluid within cleaning fluid chamber 108 has been exhausted, the apparatus is deactivated or the cleaning step is repeated with water or other cleaning fluids, as often as needed or desirable. Following this step, the apparatus of the present invention not only transfers, collects and disposes of material with minimal risk to the healthcare worker and without interruption, it also “self-cleans” all components of the apparatus by supplying a cleaning fluid to an integrated cleaning fluid chamber positioned upstream of the material transfer hose. It should be noted that in the medical, dental or surgical environment, the material interface is cleaned and sterilized in an autoclave or by other appropriate means.

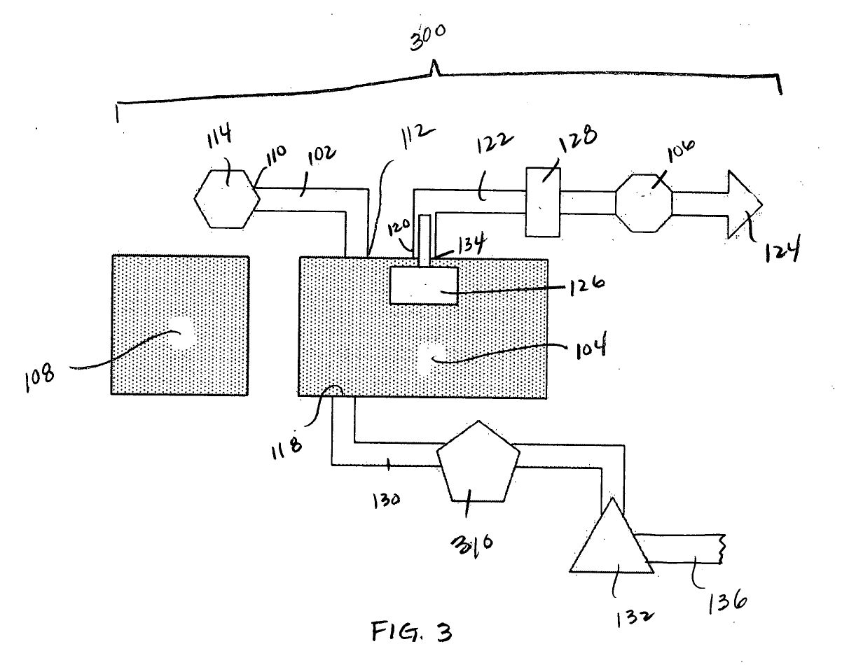

FIG. 3 illustrates an alternative embodiment of the apparatus of the present invention. Apparatus 300 is identical to apparatus 100 of FIGS. 1 and 2, except that apparatus 300 is provided with a disposal pump 310 positioned within disposal line 130 downstream of outlet 118 of material collection chamber 104 and upstream of drain 136. Disposal pump 310 is provided to continuously draw material out of material collection chamber 104 to avoid any interruptions in the material transfer process and to quickly dispose of the subject material. Disposal pump 310 may take the form of a peristaltic pump (i.e., a pump in which material is forced along by waves of contraction produced by rollers or other mechanical means on flexible tubing), such as that manufactured by Anko Products, model no. 908-108, or any other pump capable of accomplishing the objectives of the present invention. Obviously, the amount of suction drawing the material out of material collection chamber 104 should be calibrated to avoid interfering with the negative pressure generated by vacuum source 106 within material collection chamber 104, which is required to draw material into the material transfer hose of the apparatus to operate the same.

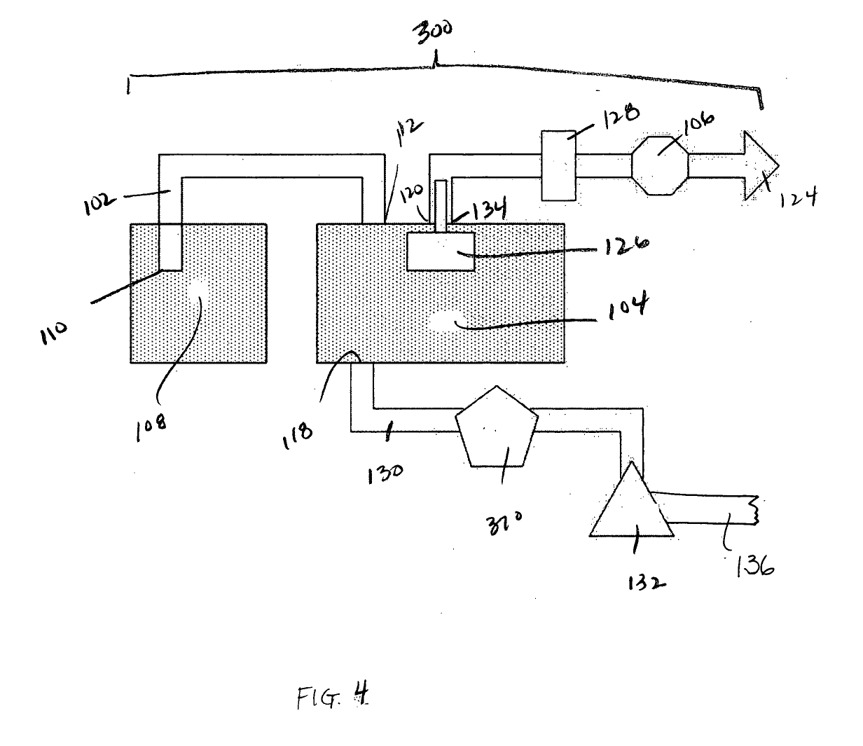

FIG. 4 illustrates the alternative embodiment of FIG. 3 in the self-cleaning configuration. Apparatus 300 is cleaned in the same manner as apparatus 100 described above by removing material interface 114, inserting material transfer hose 102 into cleaning fluid chamber 108 and activating the apparatus. In addition to cleaning material transfer hose 102, material collection chamber 104 and disposal line 130, the cleaning fluid also cleans the interior of disposal pump, as the cleaning fluid is pumped therethrough to drain 136.

Having described several embodiments of the invention, it should be realized that the apparatus of the present invention easily, safely, rapidly and continuously (i.e., without interruption) transfers, collects and disposes of a material. The present invention provides the further advantage of an integrated cleaning chamber, which can be used to clean all components of the apparatus, while utilizing relatively few parts and making maintenance of the apparatus simple.

Returning to the apparatus, both vacuum source 106 and cleaning fluid chamber 108 can be housed in a cabinet, for example an ENT cabinet known to those skilled in the art. It is also envisioned that the apparatus of the present invention could be controlled by a microprocessor provided with sensors, which automatically activates and monitors the apparatus to control the transfer, collection and disposal of a material. The microprocessor could also provide trouble shooting and over-ride features to prevent malfunctioning of the apparatus.

In instances where the apparatus is used to transfer material from differing sites on a patient, it is also envisioned that material transfer hose 102 could be provided with ports leading to multiple material interfaces adapted for the particular site or type of material transfer.

Also, and as discussed earlier, the present invention lends itself to material transfer outside of the realm of biomedical or bodily waste material. The concepts and components of the present invention can be applied to any field requiring the transfer, collection and disposal of a material, constituent or product (e.g., paint, grains, oils). It is also envisioned that the apparatus can also be used to collect and transfer a product from one area to another, but not necessarily dispose of the product. For example, the apparatus of the present invention could be used to transfer and collect paint from one area and deposit it (via the disposal line) into a container. To subsequently collect and transfer paint of a different color to another area, all components of the apparatus may be cleaned after transferring the first paint color (in accordance with the steps and structure outlined above) without contaminating the second paint color with the first.

While the invention has been particularly shown and described with reference to the preferred embodiments, it should be understood by those skilled in the art that various changes in the form and details may be made herein without departing from the scope and spirit of he invention, as defined by the following claims.

1. A self-cleaning apparatus for transferring, collecting, and disposing of waste material from a patient, comprising:
a waste material transfer hose having an inlet and an outlet for transferring waste material from the patient to the apparatus;
a waste material collection chamber having a collection chamber inlet in communication with said outlet of said waste material transfer hose and a collection chamber outlet;
a vacuum source connected to said waste material collection chamber by a vacuum line; and
a cleaning fluid chamber for receiving a cleaning fluid, said cleaning fluid chamber being arranged to communicate with and receive said inlet of said waste material transfer hose.

2. The apparatus of claim 1 further comprising:
a float movably disposed within said vacuum line to close said vacuum line to prevent waste material within said waste material collection chamber from being drawn into said vacuum line by said vacuum source, when said waste material collection chamber is filled to capacity.

3. The apparatus of claim 2 further comprising a disposal pump in communication with said collection chamber outlet of said waste material collection chamber to pump waste material out of said waste material collection chamber through said collection chamber outlet.

4. The apparatus of claim 3 further comprising a switch in electrical contact with said float and said disposal pump, such that when said float closes said vacuum line, said disposal pump is activated to dispose of waste material in said waste material collection chamber through said collection chamber outlet.

5. The apparatus of claim 4, wherein said disposal pump is a peristaltic pump connected to a drain to dispose of waste material.

6. The apparatus of claim 2 further comprising a filter disposed within said vacuum line between said vacuum source and said float to prevent waste from being drawn into said vacuum source.

7. The apparatus of claim 2, wherein said cleaning fluid chamber is positioned upstream of said inlet of said waste material transfer hose and said waste material collection chamber, to permit the cleaning of said waste material transfer hose and said waste material collection chamber.

8. The apparatus of claims 3, wherein said cleaning fluid chamber is positioned upstream of said inlet of said waste material transfer hose and said waste material collection chamber, to permit the cleaning of said waste material transfer hose, said waster material collection chamber and said disposal pump, when said cleaning fluid is drawn into said inlet of said waste material transfer hose and transferred and disposed of by said apparatus.

9. The apparatus of claim 2, wherein a waste material interface is connected to said inlet of said waste material hose, said waste material interface being a suction nozzle.

10. An apparatus for transferring, collecting and disposing of a material, comprising:
a material transfer hose having an inlet and an outlet for transferring material to the apparatus;
a material collection chamber having a collection chamber inlet in communication with said outlet of said material transfer hose and a collection chamber outlet;
a vacuum source connected to said material collection chamber by a vacuum line to draw material into said material collection chamber through said material transfer hose;
a disposal pump in communication with said collection chamber outlet to pump material out of said material collection chamber to dispose of material; and
a cleaning fluid chamber for receiving a cleaning fluid, said cleaning fluid chamber being arranged to communicate with said inlet of said material transfer hose to permit cleaning fluid to be drawn into said inlet of said material transfer hose to clean said material transfer hose, said material collection chamber and said disposal pump.

11. The apparatus of claim 10 further comprising a float movably disposed within said vacuum line to close said vacuum line to prevent material within said material collection chamber from being drawn into said vacuum line by said vacuum source when said material collection chamber is filled to capacity.

12. The apparatus of claim 11 further comprising a filter disposed within said vacuum line between said vacuum source and said float.

13. The apparatus of claim 12 further comprising a switch activated by said float, such that when said float closes said vacuum line, said disposal pump is activated to pump material out of said material collection chamber through said collection chamber outlet.

14. The apparatus of claim 10, wherein said disposal pump is a peristaltic pump in further communication with a sewer drain, such that material is disposed of in a sewer.

15. A method of cleaning a material transfer, collecting and disposal apparatus having at least a cleaning fluid chamber, a material transfer hose, a material collection chamber having an inlet in fluid communication with said material transfer hose and an outlet, and a vacuum source connected to said material collection chamber, comprising the steps of:
(a) providing said cleaning fluid chamber with a cleaning fluid;
(b) inserting said material transfer hose into said cleaning fluid chamber; and
(c) activating said vacuum source such that said cleaning fluid is drawn into said material transfer hose and said material collection chamber through said inlet of said material transfer hose to clean said material transfer hose and said material collection chamber.

16. The method of claim 15 further comprising the steps of:
(d) de-activating said vacuum source; and
(e) disposing of said cleaning fluid from said outlet of said material collection chamber after said material transfer hose and said material collection chamber have been cleaned.

17. The method of claim 16, wherein said disposing step (e) is performed by a disposal pump positioned in fluid communication with said outlet of said material collection chamber.

18. The method of claim 17 wherein said cleaning fluid cleans said disposal pump as said cleaning fluid is pumped therethrough.

19. The method of claim 18, wherein said disposal pump disposes of said cleaning fluid in a drain.

20. The method of claim 16, wherein said de-activating step (d) is performed automatically by a switch activated when said material collection chamber is filled to capacity with said cleaning fluid.
